(19) United States
(12) Patent Application Publication
(10) Pub. No.: US 2021/0290499 A1
(43) Pub. Date: Sep. 23, 2021

(54) METHOD FOR PREPARING COSMETIC COMPOSITION THROUGH ULTRA-HIGH-TEMPERATURE PASTEURIZATION

(71) Applicant: LG HOUSEHOLD & HEALTH CARE LTD., Seoul (KR)

(72) Inventors: Ki-Young KIM, Daejeon (KR); Hyeong-Jin CHO, Daejeon (KR); Hyun-Jong KANG, Daejeon (KR); Nae-Gyu KANG, Daejeon (KR)

(73) Assignee: LG HOUSEHOLD & HEALTH CARE LTD., Seoul (KR)

(21) Appl. No.: 17/259,348

(22) PCT Filed: Jul. 11, 2018
(86) PCT No.: PCT/KR2018/007874

§ 371 Date: Jan. 11, 2021

(51) Int. Cl. A61K 8/06; A61Q 19/00; A61L 2/04

(52) U.S. Cl. CPC A61K 8/06; A61Q 19/00; A61K 2800/87; A61K 2800/30; A61K 2800/805; A61L 2/04

(57) ABSTRACT

The present disclosure relates to a method for ultra-high-temperature pasteurization of a cosmetic composition using a scraped surface heat exchanger (SSHE), a method for preparing a pasteurized cosmetic composition using the method, and an ultra-high-temperature pasteurized cosmetic composition maintaining a moisture content prior to the pasteurization. According to the present disclosure, a cosmetic composition having various physical properties can be economically pasteurized in short time without change in a moisture content.

### TECHNICAL FIELD

The present disclosure relates to a method for pasteurizing a cosmetic composition.

### BACKGROUND ART

Cosmetic compositions, particularly formulations in the form of emulsion or gel, are vulnerable to microbial contamination due to high water content and long shelf life. Therefore, they usually contain preservatives such as paraben or phenoxyethanol. Recently, demand on cosmetic products not containing preservatives is increasing due to the issue of the harmful effects of the preservatives. For development of such cosmetic products, a technology of pasteurizing cosmetics is necessary.

Methods for pasteurizing cosmetics include heating at high temperature, radiation and filtration.

The method using radiation has the concern of deterioration of product quality, and the filtration method is not suitable for cosmetic compositions which are viscous or contain emulsion particles larger than microorganisms.

Ultra-high-temperature pasteurization is a method of treating cosmetics at 130-150° C. for a short period of time (several to tens of seconds) so as to increase stability as much as possible and remove even heat-resistant bacteria, and has been used mainly to improve the preservability of food products.

Korean Patent Publication No. 10-2014-0047697 discloses a preservative-free, pasteurized cosmetic product prepared using an apparatus (infusion-type ultra-high-temperature pasteurization apparatus) which infuses ultra-high-temperature steam to a cosmetic that has been heated to a specific temperature.

However, in the infusion-type ultra-high-temperature pasteurization, moisture content is changed significantly after the pasteurization because the cosmetic is directly exposed to the steam. The change in the moisture content due to the steam not only affects the feeling of the cosmetic during use but also worsens the stability of the cosmetic by changing the concentrations of its ingredients. In addition, it is not applicable to development of cosmetics having various physical properties because of the limitation in viscosity for infusion at the preheating temperature.

### DISCLOSURE

### Technical Problem

The present disclosure is directed to providing a method for ultra-high-temperature pasteurization of cosmetic compositions having various physical properties without change in moisture content, a method for preparing a pasteurized cosmetic composition using the method, and an ultra-high-temperature pasteurized cosmetic composition maintaining a moisture content prior to the pasteurization.

### Technical Solution

The present disclosure provides an ultra-high-temperature pasteurized cosmetic composition maintaining a moisture content prior to pasteurization.

The cosmetic composition according to the present disclosure is one which has been ultra-high-temperature pasteurization and maintains a moisture content prior to the pasteurization.

The phrase “maintaining a moisture content prior to the pasteurization” means that there is no substantial difference between the moisture content prior to the ultra-high-temperature pasteurization and the moisture content after the ultra-high-temperature pasteurization. The expression “no substantial difference” means that a value calculated by the following numerical formula is 2% or less, more specifically 1% or less, further more specifically 0.05% or less, most specifically 0.01% or less:

(|moisture content after ultra-high-temperature pasteurization−moisture content

prior to ultra-high-temperature pasteurization|/moisture content prior to ultra-high-temperature

pasteurization)×100(%).

The moisture content may be calculated according to various methods known in the art. For example, the moisture content may be calculated by drying the cosmetic composition before and after the ultra-high-temperature pasteurization under the same drying condition and measuring weight loss. The drying condition may be set according to the loss on drying test method. In a specific exemplary embodiment, measurement may be made after drying at 105° C. for 4 hours.

For example, “1.0% or less (1 g, 105° C., 4 hours)” means that, when about 1 g of the cosmetic composition is weighed precisely and dried at 105° C. for 4 hours, the weight loss is 10 mg or less per about 1 g.

The cosmetic composition according to the present disclosure contains moisture. The total moisture content in the cosmetic composition may be specifically 40-95 wt %, more specifically 60-85 wt %.

The cosmetic composition according to the present disclosure may have a viscosity in various ranges, and it may also be a high-viscosity cosmetic composition.

According to the present disclosure, the cosmetic composition may be pasteurized at a temperature of 130° C. or higher.

The cosmetic composition according to the present disclosure has temporal stability.

The “temporal stability” includes temporal stability under various temperature conditions, e.g., at temperatures in the range of 25 to 50° C. It includes the stability of physical properties including formulation, phase, etc.

The cosmetic composition according to the present disclosure may not contain a preservative. Specifically, the cosmetic composition to which the method according to the present disclosure is applied is substantially free from a preservative.

The expression “substantially free from” means that a preservative is contained in an amount of 1 wt % or less, 0.5 wt % or less, 0.1 wt % or less, most specifically 0 wt %, based on the total weight of the cosmetic composition.

The formulation of the cosmetic composition according to the present disclosure is not particularly limited as long as it is a formulation containing water. Examples include an O/W emulsion, a W/O emulsion, a water-dispersible gel formulation, a liquid formulation for skin, etc.

The present disclosure provides a method for ultra-high-temperature pasteurization of a cosmetic composition using a scraped surface heat exchanger (SSHE).

More specifically, the present disclosure provides a method for pasteurizing a cosmetic composition, which includes: a step of passing a cosmetic composition through a scraped surface heat exchanger (SSHE) such that an ultra-high-temperature pasteurization temperature of 130-150° C. is reached; and a step of pasteurizing the cosmetic composition as the cosmetic composition is passed through a heat insulation tube while maintaining the ultra-high-temperature pasteurization temperature.

The SSHE heat exchanger refers to a device wherein heat exchange is achieved at high speed as a scraper inside the heat exchanger rotates while scraping surface. Heat exchange is achieved without thermal shock upon contact with the wall.

The number of the SSHE heat exchanger may be one or more, more specifically 2 or more.

The cosmetic composition reaches an ultra-high-temperature pasteurization temperature (T₁) of 130-150° C. while it passes through the SSHE heat exchanger.

The scraper rotation speed of the SSHE heat exchanger for reaching the ultra-high-temperature pasteurization temperature (T₁) of 130-150° C. is 450 rpm or higher, or 450-600 rpm. If the rotation speed exceeds 600 rpm, enough heat exchange may not be achieved for a high-viscosity cosmetic composition, although the speed of heat exchange is fast. Consequently, pasteurization or cooling cannot be achieved satisfactorily. Under the premise that the pasteurization temperature is reached, the rotation speed may be adequately adjusted by those of ordinary skill within the above scraper rotation speed range in consideration of the ingredients and viscosity of the cosmetic composition, the number of the heat exchanger, etc.

The time required for passing through the SSHE heat exchanger is about 1-5 minutes although it may be different depending on the ingredients and viscosity of the cosmetic composition, the number of the heat exchanger, etc.

The pasteurization method according to the present disclosure includes the step of pasteurizing the cosmetic composition as the cosmetic composition is passed through a heat insulation tube while maintaining the ultra-high-temperature pasteurization temperature of 130-150° C. after the step of passing the cosmetic composition through the SSHE heat exchanger.

One of the purposes of passing the cosmetic composition through the heat insulation tube may be to ensure sufficient time for pasteurization.

Under the premise that enough pasteurization is possible, the shape, length, material, etc. of the heat insulation tube may be adequately selected by those of ordinary skill without limitation.

The time of passing through the heat insulation tube may be adjusted depending on the pasteurization temperature. Specifically, it may be 30 seconds or longer when the pasteurization temperature is 130° C. or higher and below 140° C., 24 seconds or longer when the pasteurization temperature is 140° C. or higher and below 150° C., and 12 seconds or longer when the pasteurization temperature is 150° C. or lower.

The time of passing through the heat insulation tube may be calculated by the following numerical formula:

time of passing through heat insulation tube(t)=(length of heat insulation tube(l)×

internal area of heat insulation tube(a))/flow speed(s)

The present disclosure also provides a method for preparing a pasteurized cosmetic composition, which includes: a step of pasteurizing a cosmetic composition according to the method described above; a step of cooling the cosmetic composition; and a step of filling the cosmetic composition in a container.

The step of cooling the cosmetic composition may be performed by various methods known in the art. Specifically, the cosmetic composition is cooled in a SSHE heat exchanger. The number of the SSHE heat exchanger for cooling the cosmetic composition is 1 or more, more specifically 2 or more.

The cooling temperature is, for example, 0-60° C., 2-58° C. or 5-55° C. If the cooling temperature is 60° C. or higher, the cosmetic composition may be deteriorated. And, if the cooling temperature is below 0° C., the cosmetic composition may be frozen.

The time required for the cooling is about 1-20 minutes, although not being limited thereto. The time may vary depending on the ingredients and viscosity of the cosmetic composition, the capacity and number of the heat exchanger, etc.

Specifically, the step of filling the container is performed in short time in order to prevent microbial recontamination. Specifically, the step of filling the container may be performed, for example, within 5 minutes to 8 hours, within 10 minutes to 7 hours, within 30 minutes to 6 hours, or within 1-5 hours.

The container may be selected by those of ordinary skill depending on the formulation, use, viscosity, etc. of the cosmetic composition.

Specifically, the method may further include, prior to the step of pasteurizing the cosmetic composition, a step of putting the cosmetic composition in a pasteurization device and transferring to the S SHE heat exchanger.

The transfer may be achieved by various methods known in the art. For example, the cosmetic composition may be transferred by a pump.

The speed of the transfer may be adjusted adequately by those of ordinary skill depending on the ingredients, viscosity, etc. of the cosmetic composition. For example, the transfer speed may be 5-60 L/h or 15-50 L/h. If the transfer speed is lower than 5 L/h, it is difficult to maintain pasteurization temperature during the pasteurization. And, if the transfer speed exceeds 60 L/h, normal operation is difficult due to increased pressure.

The method of the present disclosure may be used to pasteurize various microorganisms known to infect cosmetic compositions. For example, the microorganism to be pasteurized may be one or more selected from a group consisting of Escherichia coli, Staphylococcus aureus, Pseudomonas aeruginosa, Candida albicans, Serratia marcescens and Bacillus subtilis, although not being limited thereto.

Hereinafter, specific exemplary embodiments of the present disclosure are described referring to FIG. 1 and FIG. 2.

As an exemplary embodiment, FIG. 1 shows an ultra-high-temperature pasteurization device equipped with a scraped surface heat exchanger (SSHE) 121, 122.

The ultra-high-temperature pasteurization device shown in FIG. 1 includes an input tank 110, a heating heat exchange equipment 120, a heat insulation tube 130, a cooling heat exchange equipment 140 and a filling equipment 150.

A cosmetic composition to be pasteurized is put in the input tank 110 and then transferred by a pump 111 at a predetermined speed.

The cosmetic composition transferred to the heating heat exchange equipment 120 is reached to a pasteurization temperature (T₁). The heating heat exchange equipment includes two heat exchangers 121, 122. The heat exchangers are scraped surface heat exchangers (SSHEs).

The cosmetic composition that has passed through the heating heat exchange equipment 120 passes through the heat insulation tube 130. While passing through the heat insulation tube, it is preferred that the temperature of the cosmetic composition is maintained at the pasteurization temperature. Specifically, a temperature sensor is included at the front end and/or rear end of the heat insulation tube for this.

The cosmetic composition that has been transferred to the cooling heat exchange equipment 140 is cooled. The cooling heat exchange equipment includes two or more heat exchangers 141, 142. Like the heating heat exchange equipment, the heat exchangers are scraped surface heat exchangers (SSHEs).

The cooled cosmetic composition is transferred to the filling equipment 150 and filled in a container.

As an exemplary embodiment, FIG. 2 shows the photograph and structure of a scraped surface heat exchanger (SSHE).

### Advantageous Effects

Cosmetic compositions having various physical properties including high-viscosity cosmetic compositions can be ultra-high-temperature pasteurized without deterioration by using the method of the present disclosure.

### DESCRIPTION OF DRAWINGS

The accompanying drawings illustrate a preferred embodiment of the present disclosure and together with the foregoing disclosure, serve to provide further understanding of the technical features of the present disclosure, and thus, the present disclosure should not be construed as being limited to the drawings.

FIG. 1 shows an ultra-high-temperature pasteurization device equipped with a scraped surface heat exchanger (SSHE) 121, 122 according to an exemplary embodiment.

FIG. 2 shows the photograph and structure of a scraped surface heat exchanger (SSHE) according to an exemplary embodiment.

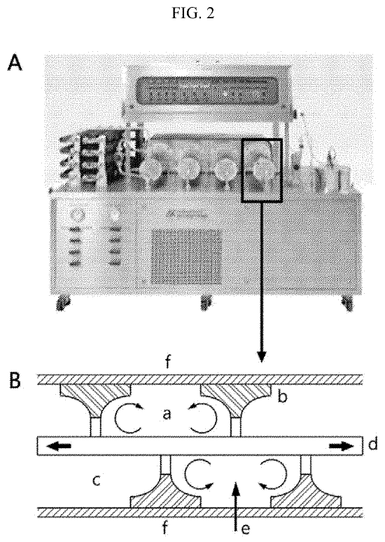

### BEST MODE

Hereinafter, the present disclosure will be described in detail with the accompanying drawings, examples, etc. However, the following examples may be changed into various other forms and it should not be construed that the scope of the present disclosure is limited by the following examples. The examples of the present disclosure are provided to the present disclosure more fully to those having ordinary knowledge in the art.

### 1. Example 1: Preparation of Cosmetic Composition

A cosmetic composition containing the ingredients described in Table 1 was prepared (unit: wt %).

TABLE 1

| Ingredients | Contents (wt %) |
|---|---|
| C₁₂₋₂₀ alkyl glucoside | 1.0 |
| PEG-100 stearate | 0.5 |
| Glyceryl stearate | 1.5 |
| Cetearyl alcohol | 2.0 |
| Polyglyceryl-3-methylglucose distearate | 1.5 |
| Hydrogenated poly decene | 5.0 |
| Tromethamine | 0.2 |
| Glycerin | 3 |
| Dipropylene glycol | 5 |
| Xanthan gum | 0.2 |
| Carbomer (Carbopol Ultrez 21, Lubrizol) | 0.2 |
| Purified water | To 100 |

### 2. Test Example 1: Establishment of Pasteurization Temperature and Temporal Condition

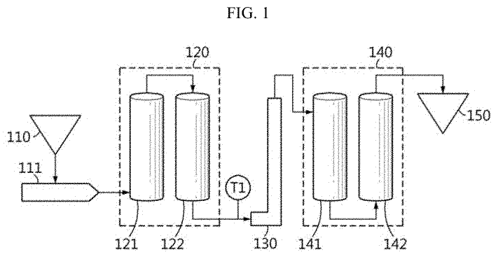

After inoculating microorganisms to the cosmetic composition of Example 1, the cosmetic composition was treated with the pasteurization device of FIG. 1 and it was investigated whether the microorganisms are killed. As microorganisms, Escherichia coli, Staphylococcus aureus, P. aeruginosa, C. albicans, Serratia marcescens and Bacillus subtilis were inoculated at 1×10⁷ CFU/g, respectively.

The flow speed of the device was set to 30 L/hour, the scraper rotation speed of heating and cooling SSHE heat exchangers was set to 600 rpm, and the internal cross-sectional area of the heat insulation tube was set to 1 cm². The pasteurization time, which is the time (t) for the composition to pass through the heat insulation tube, can be calculated from the following numerical formula: length of heat insulation tube (1)×internal area of heat insulation tube (a)/flow speed (s).

Pasteurization time(time of passing through heat insulation tube)(t)=(length of heat

insulation tube(l)×internal area of heat insulation tube(a))/flow speed(s)

The pasteurization time of the cosmetic composition of Example 1 was calculated as 12 seconds per 1 m of the heat insulation tube from the above numerical formula. After treatment with the pasteurization device under the temperature condition and time conditions described in Table 2, the number of microorganisms remaining in the medium was counted.

TABLE 2

| | Ex. 2 | Ex. 3 | Ex. 4 | Ex. 5 | Ex. 6 | Ex. 7 | Ex. 8 | Ex. 9 |
|---|---|---|---|---|---|---|---|---|
| Pasteurization | 130 | 130 | 130 | 130 | 130 | 140 | 140 | 140 |
| temperature (° C.) | | | | | | | | |
| Pasteurization time (s) | 6 | 12 | 18 | 24 | 30 | 6 | 12 | 18 |
| Number of detected | 1.2 × 10⁸ | 3.4 × 10³ | 524 | 12 | 0 | 8.2 × 10³ | 91 | 5 |
| microorganisms | | | | | | | | |
| (CFU/g) | | | | | | | | |

| | Ex. 10 | Ex. 11 | Ex. 12 | Ex. 13 | Ex. 14 | Ex. 15 | Ex. 16 |
|---|---|---|---|---|---|---|---|
| Pasteurization | 140 | 140 | 150 | 150 | 150 | 150 | 150 |
| temperature (° C.) | | | | | | | |
| Pasteurization time (s) | 24 | 30 | 6 | 12 | 18 | 24 | 30 |
| Number of detected | 0 | 0 | 2 | 0 | 0 | 0 | 0 |
| microorganisms | | | | | | | |
| (CFU/g) | | | | | | | |

As can be seen from Table 2, all the microorganisms were killed when the cosmetic composition was treated for 30 seconds or longer at 130° C., for 24 seconds or longer at 140° C., and for 12 seconds or longer at 150° C.

### 3. Test Example 2: Testing of SSHE Rpm Condition

After treating with the pasteurization device of FIG. 1 under different SSHE rpm conditions of 100-600 rpm, it was investigated whether the cosmetic composition reached the pasteurization temperature using a temperature sensor of the heat insulation tube. The pasteurization condition was fixed to 150° C. and 12 seconds.

TABLE 3

| | Ex. 17 | Ex. 18 | Ex. 19 | Ex. 20 | Ex. 21 |
|---|---|---|---|---|---|
| SSHE rpm | 100 | 150 | 300 | 450 | 600 |
| Temperature (° C.) | 117 | 131 | 142 | 150 | 150 |

As can be seen from Table 3, the pasteurization temperature (150° C.) was reached when the SSHE rpm was 450 rpm or higher.

### 4. Test Example 3: Comparison of Moisture Content Change and Temporal Stability

After ultra-high-temperature pasteurizing the composition of Table 1 under the condition of 150° C. and 12 seconds using the pasteurization device of FIG. 1 and a steam infusion-type ultra-high-temperature pasteurization device and measuring moisture content, formulation stability was observed in a constant-temperature bath of 25° C., 40° C. or 50° C. for 3 months with 1-month intervals.

Moisture content was calculated by drying the pasteurized composition at 105° C. for 4 hours. The experiment was repeated 3 times and average value was taken.

The formulation stability was evaluated by visually inspecting the surface state and the separation of oily and aqueous phases.

TABLE 4

| UHT pasteurization | | Before pasteurization | Steam infusion | SSHE |
|---|---|---|---|---|
| Moisture | Moisture content (%) | 74.3 | 79.8 | 73.9 |
| content | Change in | — | Increased by | Decreased by |
| | moisture content | | 7.4% | 0.3% |
| | after pasteurization | | | |
| Formulation | 25° C., 1 month | Good | Rough surface | Good |
| stability | 25° C., 2 months | Good | Rough surface | Good |
| | 25° C., 3 months | Good | Rough surface | Good |
| | 40° C., 1 month | Good | Rough surface | Good |
| | 40° C., 2 months | Good | Rough surface | Good |
| | 40° C., 3 months | Good | Oily phase formed | Good |
| | | | on surface | |
| | 50° C., 1 month | Good | Oily phase formed | Good |
| | | | on surface | |
| | 50° C., 2 months | Good | Oily phase | Good |
| | | | separated | |
| | 50° C., 3 months | Rough surface | Oily phase | Good |
| | | | separated | |

As can be seen from Table 4, the change in moisture content was very small for SSHE, whereas the moisture content was significantly increased for steam infusion.

In addition, for the steam infusion method, the surface was rough immediately after the treatment and the separation of the oily phase occurred after 3 months at 40° C., and after 1 month at 50° C. In contrast, for the SSHE method, the stability was similar to that before the pasteurization.

### Detailed Description of Main Elements

110: input tank,
111: pump
120: heating heat exchange equipment,
121: heat exchanger,
122: heat exchanger
130: heat insulation tube
140: cooling heat exchange equipment,
141: heat exchanger,
142: heat exchanger
150: filling equipment

### INDUSTRIAL APPLICABILITY

The present disclosure provides a method for pasteurizing a cosmetic composition.

The present disclosure provides a superior pasteurization method capable of killing microorganisms without deterioration of a cosmetic composition, and a cosmetic composition pasteurized thereby.

1. An ultra-high-temperature pasteurized cosmetic composition, maintaining a moisture content prior to the pasteurization defined by the following numerical formula within 2% or less:
(|moisture content after ultra-high-temperature pasteurization−moisture content prior
to ultra-high-temperature pasteurization|/moisture content prior to ultra-high-temperature
pasteurization)×100(%).

2. The ultra-high-temperature pasteurized cosmetic composition according to claim 1, wherein the moisture content of the cosmetic composition is 40-95 wt % based on the total weight of the composition.

3. The ultra-high-temperature pasteurized cosmetic composition according to claim 1, wherein the cosmetic composition is substantially free from a preservative.

4. The ultra-high-temperature pasteurized cosmetic composition according to claim 1, wherein the cosmetic composition is a high-viscosity cosmetic composition.

5. A method for pasteurizing a cosmetic composition, comprising: a step of passing a cosmetic composition through a scraped surface heat exchanger (SSHE) such that an ultra-high-temperature pasteurization temperature of 130-150° C. is reached; and a step of pasteurizing the cosmetic composition as the cosmetic composition is passed through a heat insulation tube while maintaining the ultra-high-temperature pasteurization temperature.

6. The method for pasteurizing a cosmetic composition according to claim 5, wherein the scraper rotation speed of the SSHE heat exchanger is 150-400 rpm.

7. The method for pasteurizing a cosmetic composition according to claim 5, Wherein a microorganism to be pasteurized is one or more selected from a group consisting of Escherichia coli, Staphylococcus aureus, Pseudomonas aeruginosa, Candida albicans, Serratia marcescens and Bacillus subtilis.

8. The method for pasteurizing a cosmetic composition according to claim 5, wherein the time of passing through the heat insulation tube is calculated by the following numerical formula:
time of passing through the heat insulation tube(t)=(length of heat insulation tube(l)×
internal area of heat insulation tube(a))/flow speed(s).

9. A method for preparing a pasteurized cosmetic composition, comprising: a step of pasteurizing a cosmetic composition according to claim 5; a step of cooling the cosmetic composition; and a step of the cosmetic composition in a container.

10. The method for pasteurizing a cosmetic composition according to claim 9, wherein the cooling is achieved by passing through a scraped surface heat exchanger (SSHE).
